(19) United States
(12) Patent Application Publication
(10) Pub. No.: US 2021/0212636 A1
(43) Pub. Date: Jul. 15, 2021

(54) TECHNOLOGIES FOR DETERMINING A CONDITION OF A PATIENT USING LC RESONATOR DATA

(71) Applicant: Hill-Rom Services, Inc., Batesville, IN (US)

(72) Inventor: Mickael Audic, Locmiquelic (FR)

(21) Appl. No.: 17/140,545

(22) Filed: Jan. 4, 2021

Related U.S. Application Data
(60) Provisional application No. 62/959,479, filed on Jan. 10, 2020.

(51) Int. Cl. A61B 5/00; A61B 5/024; A61B 5/08; A61B 5/11; A61G 7/05

(52) U.S. Cl. CPC A61B 5/6892; A61B 5/0015; A61B 5/024; A61G 2203/70; A61B 5/1115; A61G 7/05; A61B 5/08

(57) ABSTRACT

A flexible mat may be configured to be placed between a patient and a patient support apparatus. The flexible mat may be used for determining a condition of a patient based on LC resonator data. The flexible mat may include a set of one or more LC resonators. Each LC resonator may include a capacitor and an inductor and may be configured to oscillate at a frequency associated with a present capacitance of the capacitor. The flexible mat may also include circuitry configured to measure an oscillation frequency of at least one of the LC resonators. The circuitry may also be configured to convert the measured oscillation frequency to digital frequency data which may be indicative of the measured oscillation frequency. The circuitry may also be configured to determine, based on the frequency data, at least one of a position, a heart rate, or a respiration rate of the patient.

### CROSS-REFERENCE TO RELATED APPLICATION

This application claims the benefit, under 35 U.S.C. § 119(e), of U.S. Provisional Patent Application No. 62/959,479, filed Jan. 10, 2020, the entirety of each of which is hereby expressly incorporated by reference herein.

### BACKGROUND

The present disclosure relates to determining a condition of a patient and particularly, to using capacitors to determine a condition of a patient on a patient support apparatus.

In a typical hospital setting, one or more patients may be located in each room of the hospital and may be periodically checked on by caregivers (e.g., nurses, doctors, etc.). If a patient is in need of assistance when a caregiver is not in the patient's room, the patient may affirmatively request assistance, such as by pressing a call button located on or near the patient's bed (e.g., patient support apparatus), which may send a corresponding request to a nurse call system operated in the hospital. Patients may be monitored by one or more devices that measure physiological conditions of the patient, such as heart rate, blood pressure, and/or other conditions. If a monitored condition does not satisfy a predefined target condition (e.g., if the monitored heart rate is outside of a predefined range), a device may automatically initiate a request to the nurse call system. However, such devices typically require physical contact with the patient, which the patient may find intrusive. While some other devices may measure physiological conditions of a patient without physically contacting the patient, they are particularly sensitive to noise (e.g., electromagnetic interference) which may reduce their accuracy and/or require additional cost and complexity to compensate for the noise.

### SUMMARY

The present application discloses one or more of the features recited in the appended claims and/or the following features which, alone or in any combination, may comprise patentable subject matter:

According to an aspect of the present disclosure, a flexible mat may be configured to be placed between a patient and a patient support apparatus. The flexible mat may be used for determining a condition of a patient based on LC resonator data. The flexible mat may include a set of one or more LC resonators. Each LC resonator may include a capacitor and an inductor and may be configured to oscillate at a frequency associated with a present capacitance of the capacitor. The flexible mat may also include circuitry configured to measure an oscillation frequency of at least one of the LC resonators. The circuitry may also be configured to convert the measured oscillation frequency to digital frequency data which may be indicative of the measured oscillation frequency. The circuitry may also be configured to determine, based on the frequency data, at least one of a position, a heart rate, or a respiration rate of the patient.

In some embodiments, the flexible mat may obtain frequency data from multiple LC resonators. Further, in determining the position of the patient based on the obtained frequency data, the circuitry in the flexible mat may be configured to convert the frequency data to capacitance data for each LC resonator. The circuitry may also be configured to determine a difference in capacitance between capacitors of multiple LC resonators and determine the position of the patient as a function of the difference in the capacitance. The flexible mat, in some embodiments, may determine the difference in capacitance between capacitors that are adjacent to each other in the mat. Additionally or alternatively, the circuitry in the flexible mat may determine a difference in capacitance of one of the capacitors over time. The circuitry, in some embodiments, may determine whether the patient is located on the patient support apparatus, whether the heart rate satisfies a reference heart rate, and/or whether the respiration rate satisfies a reference respiration rate. The circuitry may also provide data indicative of the patient's position, heart rate, and/or respiration rate to another device.

The LC resonators of the flexible mat may be formed by two arrays of conductive plates on opposite sides of a dielectric sheet. In some embodiments, the circuitry may obtain frequency data from each of eight LC resonators. One or more of the LC resonators in the flexible mat may be formed by a set of capacitive plates configured as a single ended grounded sensor. Additionally or alternatively, one or more of the LC resonators may have capacitive plates configured as a differential grounded sensor, a single ended floating sensor or a differential floating sensor. In some embodiments, the circuitry of the flexible mat may obtain frequency data indicative of a shift in a resonant frequency of one or more of the LC resonators. The flexible mat may obtain frequency data that may be indicative of a change in a distance between two plates of a capacitor of one of the LC resonators in the flexible mat. Additionally or alternatively, the flexible mat may obtain frequency data that may be indicative of a change in an electric field between two adjacent capacitive plates on the same side of a dielectric sheet in the mat.

In another aspect of the present disclosure, a patient bed may include a frame that has a support deck. The patient bed may also include a mattress and a flexible mat located between the support deck and the mattress. The flexible mat may include a set of one or more LC resonators. Each LC resonator may include a capacitor and an inductor and may be configured to oscillate at a frequency associated with a present capacitance of the capacitor. The flexible mat may also include circuitry configured to measure an oscillation frequency of at least one of the LC resonators. The circuitry may also be configured to convert the measured oscillation frequency to digital frequency data that may be indicative of the measured oscillation frequency and may determine, based on the frequency data, at least one of a position, a heart rate, or a respiration rate of the patient.

In some embodiments, the circuitry in the flexible mat may obtain frequency data from multiple LC resonators and, in determining, based on the obtained frequency data, a position of the patient, the circuitry in the flexible mat may convert the frequency data to capacitance data for each LC resonator. The circuitry may also determine a difference in capacitance between capacitors of multiple LC resonators and may determine the position of the patient as a function of the difference in the capacitance. The flexible mat, in some embodiments, may determine a difference in capacitance between adjacent capacitors.

In yet another aspect of the present disclosure, a method may be performed by a flexible mat located between a patient and a patient support apparatus. The flexible mat may include a set of one or more LC resonators and each LC resonator may include a capacitor and an inductor and may be configured to oscillate at a frequency associated with a present capacitance of the capacitor. The method may include measuring, by the flexible mat, an oscillation frequency of at least one of the LC resonators. Additionally, the method may include converting, by the flexible mat, the measured oscillation frequency to digital frequency data that may be indicative of the measured oscillation frequency. Further, the method may include determining, by the flexible mat and based on the frequency data, at least one of a position, a heart rate, or a respiration rate of the patient.

In another aspect of the present disclosure, one or more computer-readable storage media may include instructions that, when executed, may cause a flexible mat to perform a set of operations. The flexible mat may be located between a patient and a patient support apparatus and may have a set of one or more LC resonators. Each LC resonator may include a capacitor and an inductor and may be configured to oscillate at a frequency associated with a present capacitance of the capacitor. The instructions may cause the flexible mat to measure an oscillation frequency of at least one of the LC resonators. The instructions may also cause the flexible mat to convert the measured oscillation frequency to digital frequency data that may be indicative of the measured oscillation frequency. Additionally, the instructions may cause the flexible mat to determine, based on the frequency data, at least one of a position, a heart rate, or a respiration rate of the patient.

Additional features, which alone or in combination with any other feature(s), such as those listed above and/or those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of at least one embodiment of a hospital bed suitable for use with embodiments of a mat for determining a condition of a patient using LC resonator data;

FIG. 2 is a top plan view of a patient support deck of the hospital bed of FIG. 1;

FIG. 3 is a perspective view of at least one embodiment of the mat for determining a condition of a patient using LC resonator data;

FIG. 4 is a perspective view of the mat in situ on the patient support deck of the hospital bed of FIGS. 1 and 2;

FIG. 5 is a simplified elevation view showing a spatial relationship between a patient resting on a hospital bed and capacitive plates of the mat of FIG. 3;

FIG. 6 is a simplified schematic view of circuitry utilized by the mat of FIG. 3 to determine a condition of a patient;

FIG. 7 is a schematic view of an LC resonator circuit that may be used in the mat of FIG. 3 to determine a condition of a patient;

FIGS. 8-11 are schematic views of different sensor configurations that may be used in the mat of FIG. 3 to determine a condition of a patient;

FIG. 12 is an exploded view of components of the mat of FIG. 3;

FIG. 13 is an exploded view of a spatial relationship between a human body and the components of the mat shown in FIG. 12;

FIG. 14 is a simplified view of an electric field between plates of the mat when a human body is not positioned above the plates;

FIG. 15 is a simplified view of an electric field between the plates of the mat when a human body is positioned above the plates; and

FIGS. 16-18 are simplified flow diagrams of at least one embodiment of a method for determining a condition of a patient that may be performed by the mat of FIG. 3.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

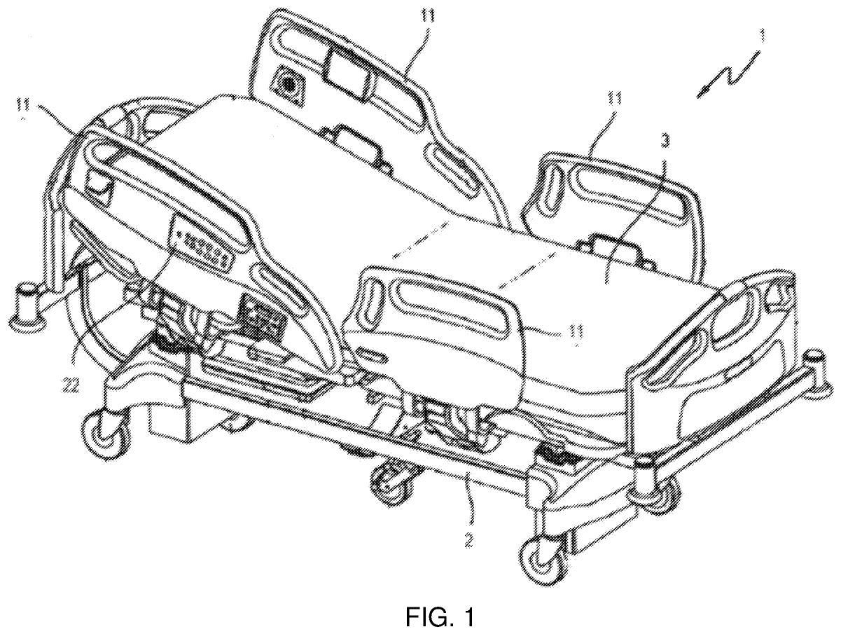

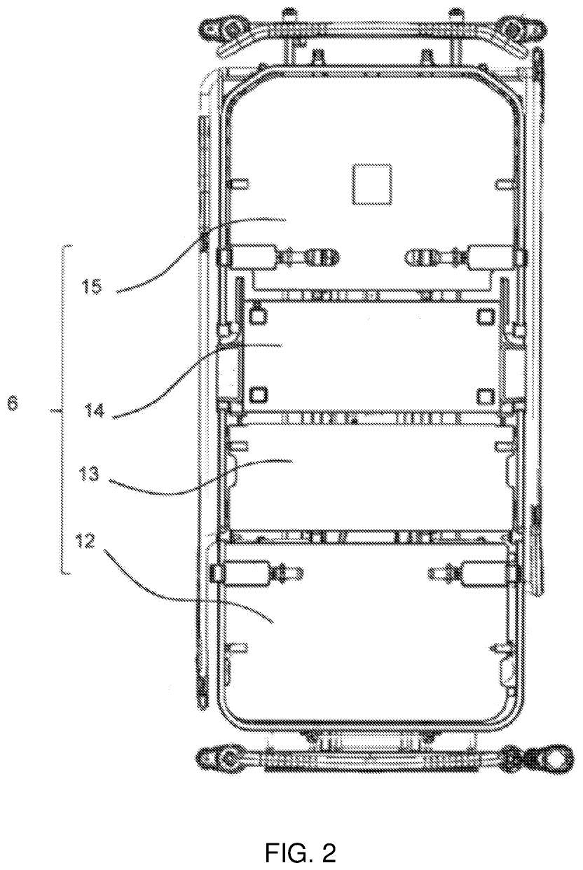

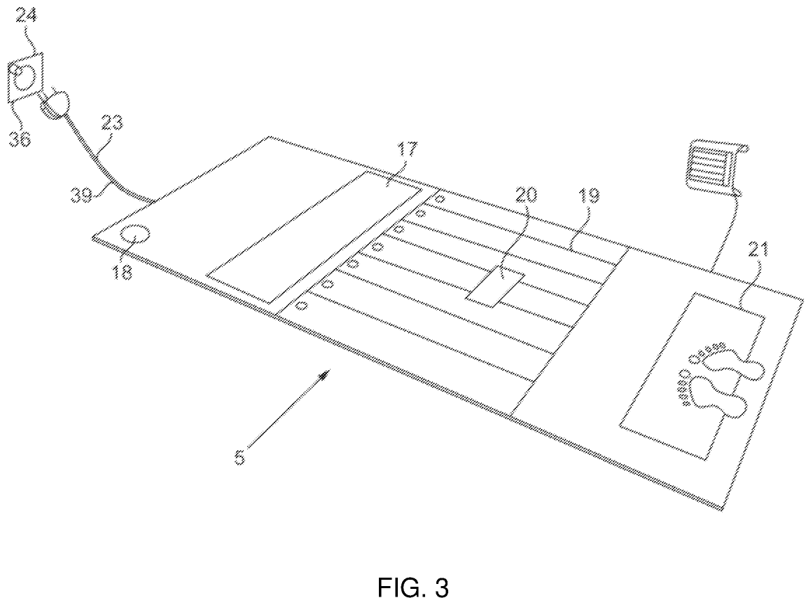

Referring now to FIG. 1, a patient support apparatus 1, includes a frame 2 and a patient support surface 3, such as a mattress. As can be seen, in FIG. 1, the patient support apparatus is embodied as a hospital bed. However, the patient support apparatus 1 may alternatively be embodied as a stretcher or any other apparatus capable of physically supporting all or a portion of a patient's body. In the illustrative embodiment, a flexible mat 5 (see FIG. 3) is located between the mattress 3 and a support deck 6 (see FIG. 2) of the patient support apparatus 1. The frame 2 includes a lower frame (aka a base frame), supports or lift mechanisms coupled to the lower frame, and an upper frame movably supported above the lower frame by the supports. The lift mechanisms may be configured to raise and lower the upper frame with respect to the lower frame and move the upper frame between various orientations, such as Tredelenburg and reverse Trendelenburg.

The upper frame carries the support deck 6 and a set of siderails 11. The illustrative deck 6 includes a leg section 12, a thigh section 13, a seat section 14, and a head and torso section 15, as shown in FIG. 2. The leg section 12 and the thigh section 13 define a lower limb support section. The head and torso section define an upper body support section. The leg section 12, the thigh section 13, and the seat section 14 define a lower body support section. The siderails 11 are configured to move between a deployed position and a storage position, and may be used to locate the perimeter of the upper frame and assist with ingress to the patient support apparatus 1 and egress from the patient support apparatus 1.

The patient support surface 3 (e.g., mattress), in the illustrative embodiment, is configured to support a person (e.g., a patient) thereon and move with the deck 6 between the various configurations. Further, in the illustrative embodiment, the patient support surface 3 includes a leg portion, a thigh portion, a seat portion, and a head and torso portion, which are each supported on corresponding sections 12, 13, 14, 15 of the deck 6.

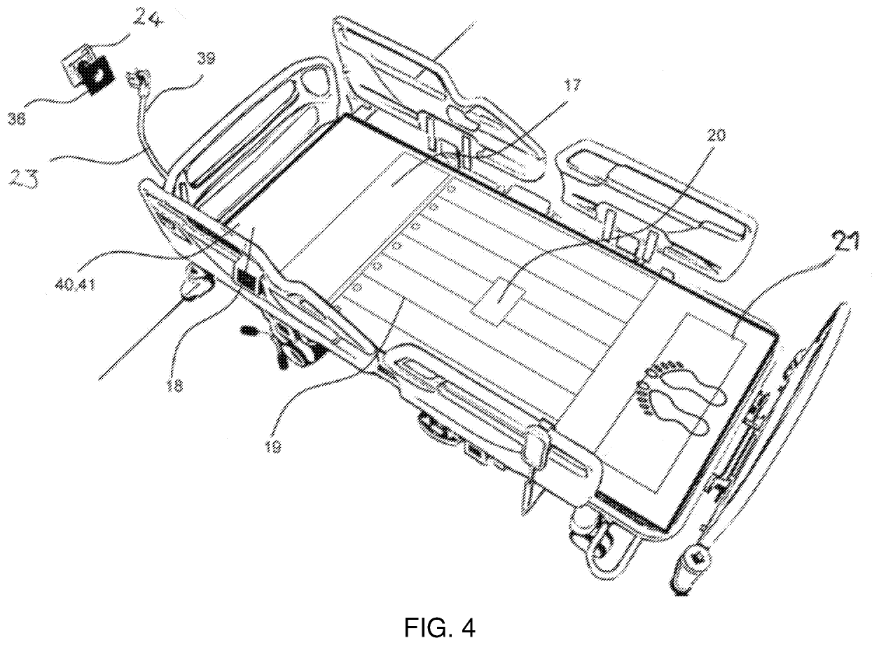

Referring to FIGS. 3 and 4, the flexible mat 5 includes sensors 17, 19, 20 to monitor conditions of a patient. A bed or frame sensor 18 for monitoring one or more characteristic's of the configuration of the patient support apparatus 1 (e.g., hospital bed) is located on the flexible mat 5. The sensors 17, 19, 20 monitor a patient's position and movement, a patient's respiration rate, a patient's heart rate, and/or other conditions (e.g., temperature). A data processor 21 (e.g., mat control unit) includes communication circuitry (e.g., wireless or wired communication circuitry, such as a network interface controller, a wireless network interface controller, etc.) for communication with a hospital data network, in some embodiments. In some embodiments, outputs of the sensors are transmitted to the data processor 21.

In some embodiments, a caregiver bed or patient support apparatus control graphical user interface (GUI) 22 is located on an outboard side of one of the siderails 11. The GUI 22 may include bed position adjustment controls, such as head up and down controls, leg up and down controls, chair positioning controls, Trendelenburg and reverse Trendelenburg controls, and bed up and down controls. In some embodiments, one or more of the above controls are manual (e.g., physical) controls, such as buttons, levers, or switches, rather than graphical controls on the GUI 22.

Referring to FIG. 3, the flexible mat 5 is made, for example, from a textile or nylon mesh coated with polyurethane on which various sensors and associated circuitry are mounted. The mat 5, in the illustrative embodiment, is relatively thin (e.g., 4-5 millimeters), with a thicker section (e.g., 20 millimeters) sized to support the data processor 21 mounted thereon. Due to its thinness and flexibility, the mat 5, in the illustrative embodiment, may be rolled up for transport and/or storage when it is not in use on a patient support apparatus (e.g., on the hospital bed 1). The mat 5, in the illustrative embodiment, is sized to snugly align with a standard patient support deck. In some embodiments, the patient support deck 6 includes upstanding edges that can hold the mat 5 in place. A standard hospital mattress is about 200 centimeters long and about 90 centimeters wide. The patient support deck 6 is slightly larger to accommodate that mattress size.

The mat 5 may, in addition or alternatively to the alignment provided by the snug fit, include fasteners to fix and/or align the mat 5 on the deck 6. The fasteners, in some embodiments, include at least one of a hook and loop fasteners (e.g., Velcro) and straps to fix the mat 5 to the frame or deck 6. The mat 5 may be sized and otherwise adapted to occupy a predetermined position and orientation on the deck 6 so that the location of at least its frame characteristic sensors are in a predetermined location relative to the frame and support deck 6. At least some of the sensors of the mat 5 may be at selected positions relative to the longitudinal axis (i.e., length) of the bed 1 so the key alignment is in the longitudinal direction. The mat 5 may therefore be dimensioned so that it is held in place along that longitudinal axis by virtue of being adjacent to the footboard and headboard at the respective foot and head ends of the bed 1.

As described above, the illustrative mat 5 includes multiple different sensors, including sensors 17, 19, 20 for sensing one or more conditions of a patient and a bed or frame sensor 18 for sensing a condition of the frame or bed 1. The sensor 17 is a capacitive or piezo-electric pressure sensing array 17 at the head end of the mat 5, in some embodiments. The sensor 17 is used to monitor one or more of a respiratory rate, a heart rate, and a sleep condition of a patient on a mattress above the mat 5. The sensor 19 is used to monitor the position of the patient. As described herein, in some embodiments, the sensor 19 may also perform some of the sensing functions (e.g., respiratory rate and/or heart rate sensing) of the sensor 17 (e.g., in lieu of the sensor 17). The sensor 20 may include an incontinence detection antenna for communication with a moisture detection element placed on the upper surface of the mattress. The sensor 18 includes an accelerometer for monitoring the head of bed (“HOB”) angle of the head of the bed frame, in some embodiments.

The data processor 21 is in communication with each of the sensors 17, 18, 19, 20 on the mat 5 and with one or more locations (e.g., compute devices) remote from the mat 5 and the bed 1 on which the mat 5 is placed. The data processor 21 may include wireless and/or wired communication circuitry that communicatively couples the data processor 21 to the remote location(s) (e.g., compute device(s)). The remote location(s) may include at least one of a hospital information network, nurse station computer monitor, ward status board, hallway alarm, call light, and/or a mobile device (e.g., computer, phone, pager, etc.).

The mat 5 and its associated sensors and electronics is powered by a power line 23 for wired connection to a power socket 24 (e.g., in the wall of the hospital ward or room in which the bed 1 is located), in the illustrative embodiment. The sensors are therefore in wired communication with the power source accessed via the power line 23. Alternatively or additionally, the mat 5 may include a transmitter which may be detectably connected to the bed power and communication by which to provide a wireless power source for one or more of the sensors. A battery may also be provided on the mat 5 as a primary or back-up power source, if desired. That battery may be charged by the transmitter or some other wireless charging system.

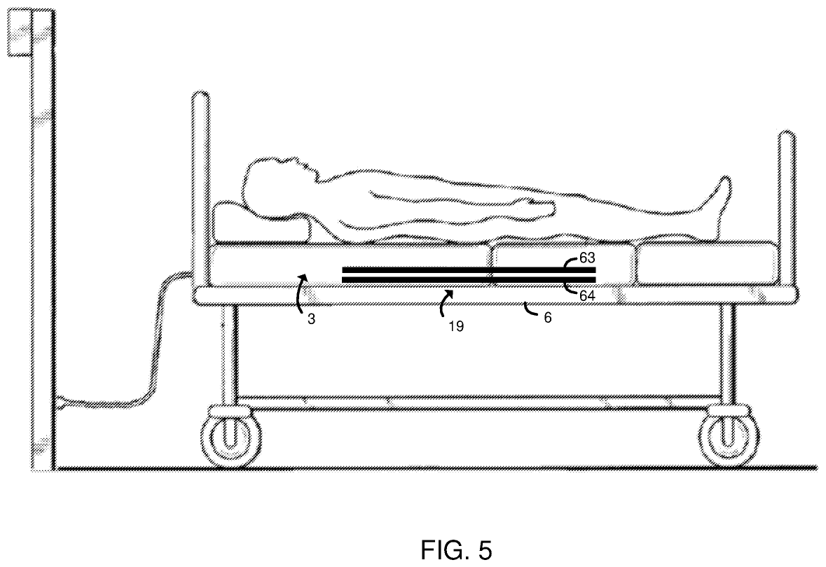

Referring now to FIG. 5, the sensor 19 generally includes at least one set of capacitive plates 63, 64 that, as described in more detail herein, form one or more capacitors of corresponding LC resonator circuits (also referred to herein as “LC resonators”). Changes in the capacitance of the plates 63, 64 (e.g., caused by a change in the distance between the plates 63, 64 when a patient is laying on the mattress and forcing the plates 63, 64 closer together) results in a change in the oscillation frequency (e.g., resonant frequency) of the corresponding LC resonator(s). In FIG. 5, plates 63, 64 are shown within mattress 3 for ease of illustration but it should be understood that plates 63, 64 are included in the mat 5 located between mattress 3 and deck 6.

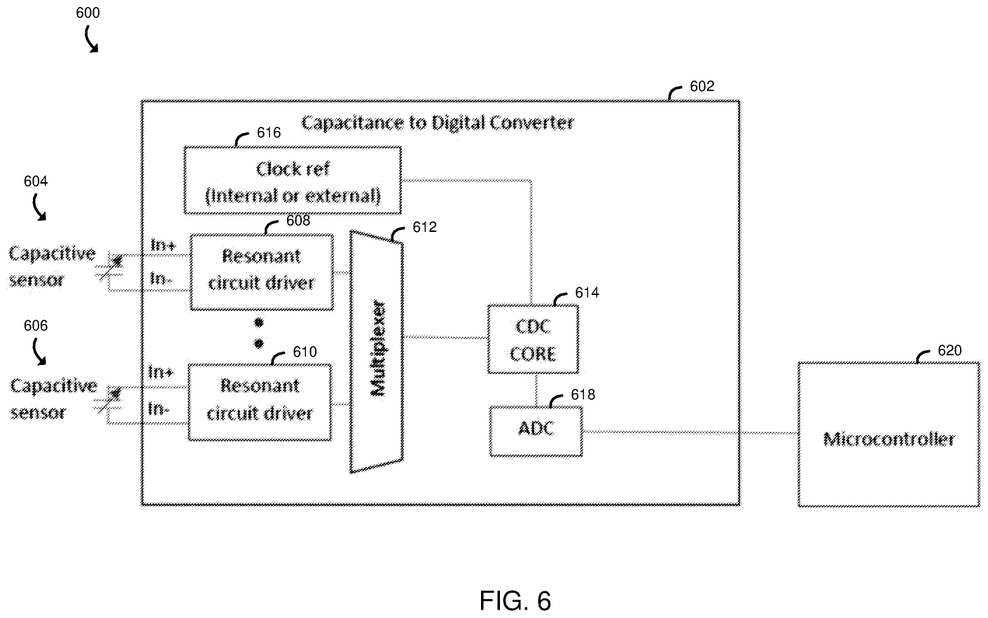

Referring now to FIG. 6, circuitry 600 used in the mat 5 (e.g., in the sensor 19 and the data processor 21) for detecting condition(s) (e.g., presence, position, heart rate, and/or respiration rate) of a patient, in the illustrative embodiment, include a capacitance to digital converter (CDC) 602 coupled to a set of capacitive sensors 604, 606 (e.g., each formed by capacitive plates, similar to the plates 63, 64 of FIG. 5). As described in more detail herein, the plates are arranged in substantially co-planar arrays that are separated by a dielectric material (e.g., a dielectric sheet), in some embodiments. Each capacitive sensor 604, 606 is connected to a corresponding resonant circuit driver 608, 610, each of which may be embodied as circuitry (e.g., an inductor) to cause a current to charge and/or recharge the capacitor (e.g., capacitive sensor 604, 606) with voltages of alternating polarity as the LC resonator oscillates.

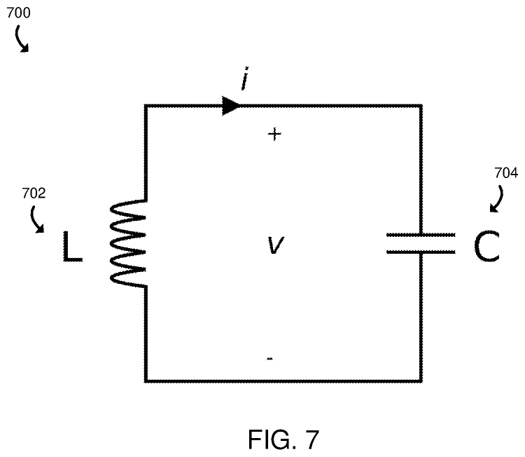

Referring briefly to FIG. 7, a schematic diagram of an LC resonator 700 is shown. The LC resonator 700 is representative of the combination of the capacitive sensor 604 and the resonant circuit driver 608, and also of the combination of the capacitive sensor 606 and the resonant circuit driver 610. In the LC resonator 700, the capacitor 704 stores energy in an electric field between the plates, depending on the voltage across the capacitor 704 and the inductor 702 stores energy in its magnetic field, depending on the current passing through the inductor 702. The voltage across the capacitor 704 drives a current through the inductor 702, which builds up a magnetic field around the inductor 702. The voltage across the capacitor 704 decreases to zero as the charge is consumed by the current. Subsequently, the energy stored in the magnetic field of the inductor 702 induces a voltage across the inductor 702. The induced voltage causes a current to recharge the capacitor 704 with a voltage of opposite polarity to the previous voltage across the capacitor 704 and so on.

The LC resonators 700 used in the mat 5 (e.g., in the circuitry 600 of the sensor 19) have a narrow-band architecture that provides protection from electromagnetic interference (EMI) and greatly reduces the noise floor compared to typical RC (resistor-capacitor) circuits. A change in the capacitance of the capacitor 704 (e.g., the capacitive sensor 604, which is formed by plates 63, 64, as described above) changes the frequency of oscillation (resonant frequency) of the LC resonator. Referring back to FIG. 6, the resonant circuit drivers 608, 610 are connected to a multiplexer 612 which is connected to a capacitance to digital converter (CDC) core 614. The CDC core 614 is also connected to a reference clock signal 616 (e.g., a signal that represents a predefined reference frequency). The CDC core 614 may be embodied as any circuitry configured to measure the oscillation frequency (resonant frequency) of each of the LC resonators (e.g., capacitive sensor 604 and resonant circuit driver 608, capacitive sensor 606 and resonant circuit driver 610). The CDC core 614 is connected to analog to digital converter 618 which may be embodied as any device or circuitry configured to convert an analog value (e.g., a voltage) to a corresponding digital value (e.g., a set of one or more bits indicative of a number).

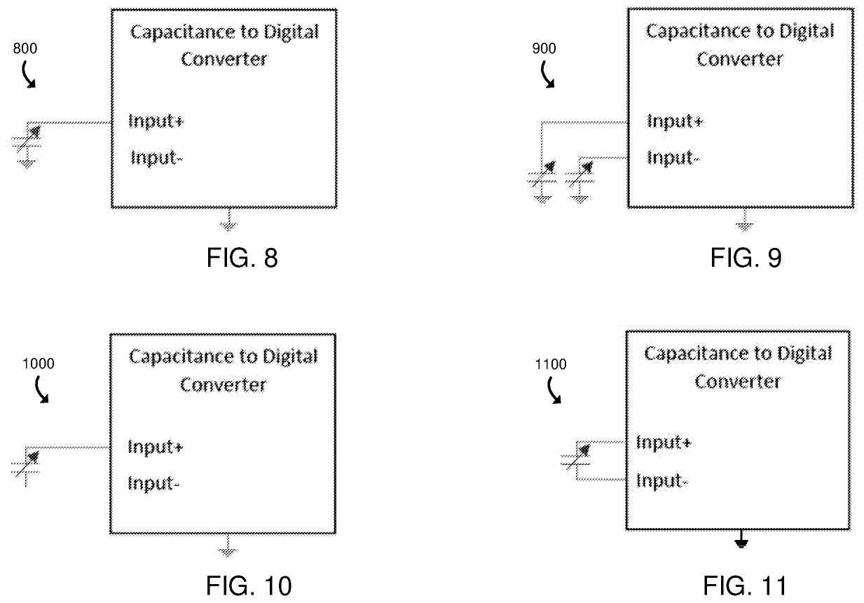

In operation, for each LC resonator, the capacitance to digital converter 602 outputs (e.g., to a microcontroller 620, which may be connected to or embodied as the data processor 21) a digital value that is proportional to frequency (e.g., represents a measurement of the resonant frequency of each LC resonator). The frequency measurement may be converted (e.g., by the microcontroller 620) to an equivalent capacitance. The microcontroller 620 may detect differences between resonant frequencies (or capacitance values) of the LC resonators over time and between the LC resonators to determine whether a patient is present on the bed 1 or has left the bed 1, the location of the patient on the bed 1, the heart rate, and/or the respiration rate of the patient. Referring briefly to FIGS. 5 and 8-11, the capacitive plates 63, 64 forming a capacitive sensor (e.g., the capacitive sensor 604 or capacitive sensor 606) may be embodied as a single ended grounded sensor 800, a differential grounded sensor 900, a single ended floating sensor 1000, or a differential floating sensor 1100.

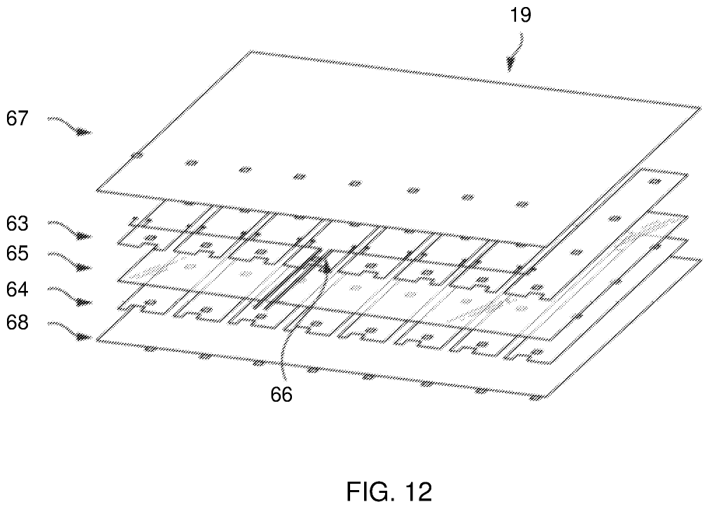

Referring now to FIG. 12, the sensor 19 may extend through a thigh section and torso section of the mat 105. As shown in FIG. 12, the sensor 19 includes an array of capacitive pressure sensing elements, formed by plates 63, 64. In the illustrative embodiment, the sensor 19 includes eight pressure sensing elements (e.g., capacitors formed by pairs of capacitive plates 63, 64), each pressure sensing element extending substantially the length of the mat 105. Accordingly, each pressure sensing element is arranged to extend longitudinally along the support deck 6 of the patient support apparatus 1 (e.g., hospital bed). The capacitive sensing elements are arranged in a row extending substantially the width of the flexible mat 5.

In more detail, the illustrative sensor 19 includes an array of eight upper capacitive plates 63 formed from an electrically conductive fabric, such as nickel/copper (NiCu) coated nylon. Each plate 63 illustratively has a width of about 85 millimeters, a length of about 710 millimeters, and a thickness of about 0.13 millimeters. Adjacent plates 63 are spaced apart with a gap of about 15 millimeters between them. The illustrative sensor 19 also includes eight lower capacitive plates 64, similar to the upper capacitive plates 63. Each lower capacitive plate 64 is aligned below a corresponding one of the upper capacitive plates 63. A relatively thin dielectric sheet 65, which may be formed from any dielectric material (e.g., polyurethane (PU)), is illustratively positioned between the upper capacitive plates 63 and the lower capacitive plates 64, such that each pair of upper and lower capacitive plates 63, 64 forms a capacitor. The dielectric sheet 65 illustratively has a thickness of about 320 micrometers and extends substantially across the length and width of the sensor 19. Pairs of wires 66, in the illustrative embodiment, are connected to each capacitor, with a first wire being connected to the upper capacitive plate 63 and the second wire being connected to the lower capacitive plate 64 of each capacitor.

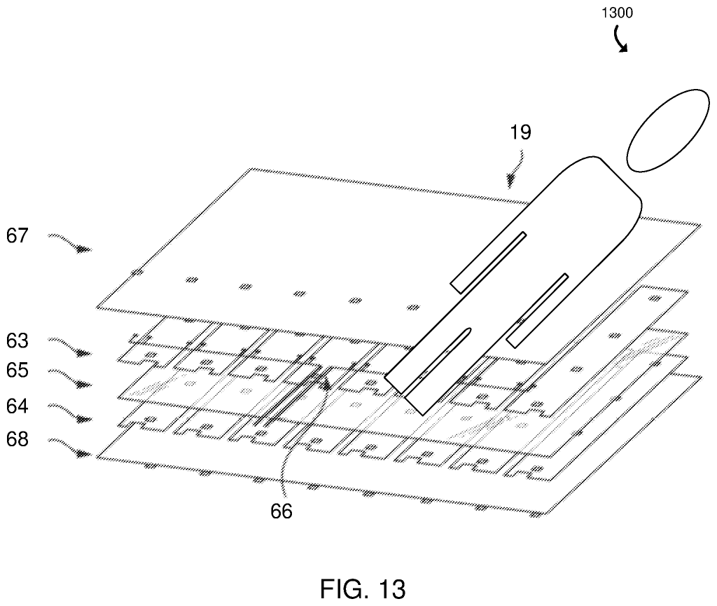

The capacitive plates 63, 64 and the dielectric sheet 65 are enclosed between a polyurethane top cover 67 and a similar bottom cover 68. The top cover 67 and the bottom cover 68 may be welded together at the edges. Each capacitor (e.g., pair of upper plate 63 and lower plate 64) is connected to the CDC 602 (shown in FIG. 6, each as a capacitive sensor 604, 606). For example, the top plate 63 may be connected to a positive input of the CDC 602 and the lower plate 64 may be connected to a negative input of the CDC 602 in the differential floating sensor configuration 1100 shown in FIG. 11. As discussed above, other configurations 800, 900, 1000 are also possible (e.g., with one or more of the plates 63, 64 connected to ground or floating). Further, and as discussed above, when the distance between the plates 63, 64 of any capacitive sensor 604, 606 changes (e.g., due to pressure or lack of pressure from a human body), the capacitance changes correspondingly, as does the oscillation frequency (resonant frequency) of the corresponding LC resonator. As such, and with reference to FIG. 13, the mat 5 (e.g., the circuitry 600) may identify the LC resonators having different resonant frequencies than the other LC resonators and determine that those LC resonators having the different resonant frequencies correspond with capacitors defined by plates 63, 64 that have been pressed closer together due to the presence of a human body 1300 (e.g., a patient) on the mattress 3 of the patient support apparatus 1 (e.g., hospital bed).

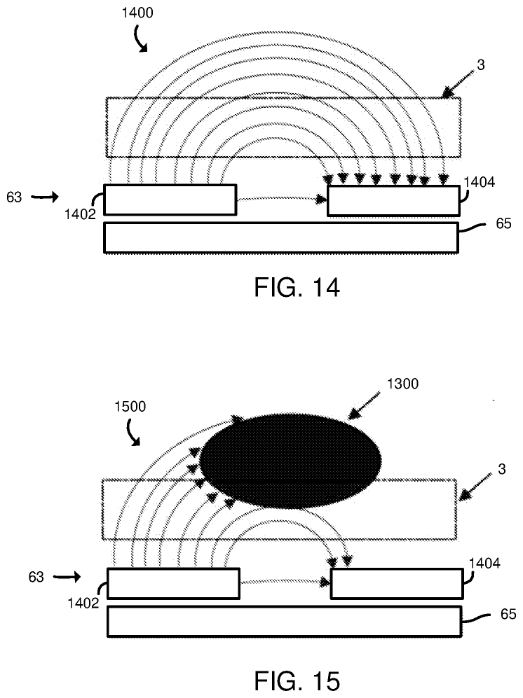

In addition to causing a change in capacitance due to the weight of the human body pressing plates 63, 64 closer together, the human body 1300 also affects the ability of an electromagnetic field to pass from one plate to an adjacent plate in the same horizontal plane. Referring now to FIG. 14, in the absence of the human body 1300 (i.e., patient) on the mattress 3, an electromagnetic field is transmitted from one plate 1402 to another plate 1404 of the set of plates 63 (e.g., the upper plates) without interference from the human body 1300. By contrast, in FIG. 15, when the human body 1300 is present on the mattress 3, the electric field 1500 between the two plates 1402, 1404 is modified. The modification to the electric field 1500 affects the capacitance of the capacitor associated with the plate 1404 and the resulting resonant frequency of the corresponding LC resonator. As such, by detecting a difference in resonant frequencies between LC resonators having adjacent capacitive plates (e.g., the plates 1402, 1404), the mat 5 (e.g., the circuitry 600) is able to determine that a human body (e.g., patient) is present on the mat 5, and the location of the human body (e.g., by determining which of the LC resonators have modified resonant frequencies).

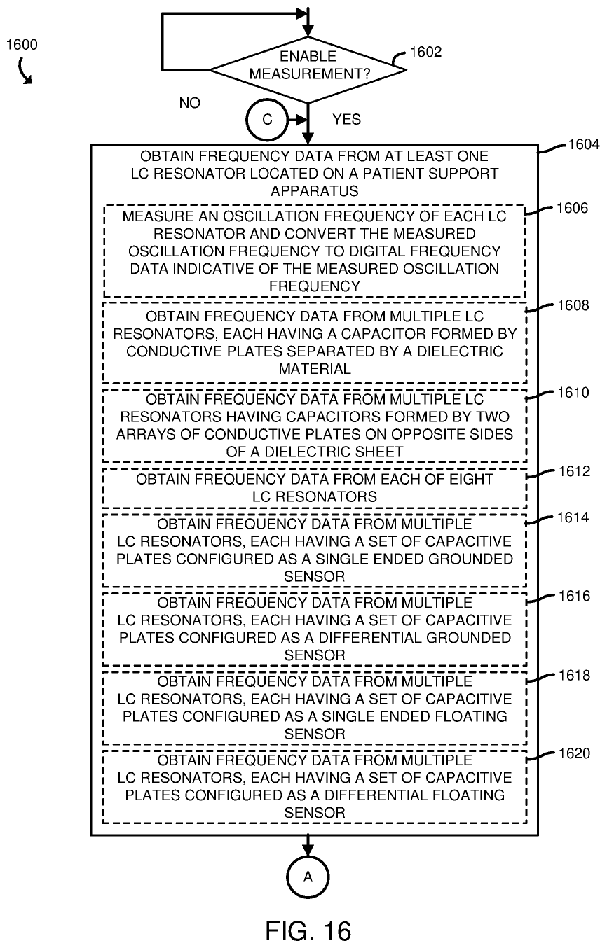

Referring now to FIG. 16, the mat 5, in operation, performs a method 1600 of measuring capacitance to determine a condition of a patient. The method 1600 begins with block 1602 in which the mat 5 determines whether to enable measurement. In the illustrative embodiment, the mat 5 determines to enable measurement if it is equipped with the sensor 19. In other embodiments, the mat 5 may determine to enable measurement based on additional factors (e.g., in response to a request to the data processor 21 (e.g., from a remote compute device) to enable measurement, etc.). Regardless, in response to a determination to enable measurement, the method 1600 advances to block 1604, in which the mat 5 obtains frequency data from at least one LC resonator (e.g., the combination of the capacitive sensor 604 and the resonant circuit driver 608, the combination of the capacitive sensor 606 and the resonant circuit driver 610, etc.) located on a patient support apparatus (e.g., the mat 5 is supported on the deck 6 of the patient support apparatus 1 (e.g., hospital bed)).

As indicated in block 1606, the mat 5 measures an oscillation frequency (e.g., resonant frequency) of each LC resonator and converts the measured oscillation frequency to digital frequency data (e.g., convert an analog value to a digital value) indicative of the measured oscillation frequency of each LC resonator. In obtaining frequency data, and as indicated in block 1608, the mat 5 obtains frequency data from multiple LC resonators, each having a capacitor formed by conductive plates (e.g., plates 63, 64) on opposite sides of a dielectric sheet (e.g., the dielectric sheet 65). As indicated in block 1610, in obtaining frequency data, the mat 5 obtains frequency data from multiple LC resonators having capacitors formed by two arrays of conductive plates (e.g., an array of upper plates 63 and an array of lower plates 64) on opposite sides of a dielectric sheet (e.g., the dielectric sheet 65). In some embodiments, the mat 5 may obtain frequency data from each of eight LC resonators, as indicated in block 1612.

As indicated in block 1614, in obtaining the frequency data, the mat 5 obtains frequency data from one or more LC resonators that each have conductive plates configured as a single ended grounded sensor (e.g., the single ended grounded sensor 800). Additionally or alternatively, and as indicated in block 1616, the mat 5 may obtain frequency data from one or more LC resonators that each have conductive plates configured as a differential grounded sensor (e.g., the differential grounded sensor 900). In some embodiments, and as indicated in block 1618, the mat 5 may obtain frequency data from one or more LC resonators that each have a set of capacitive plates configured as a single ended floating sensor (e.g., the single ended floating sensor 1000). Additionally or alternatively, the mat 5 may obtain frequency data from one or more LC resonators that each have a set of capacitive plates configured as a differential floating sensor (e.g., the differential floating sensor 1100), as indicated in block 1620.

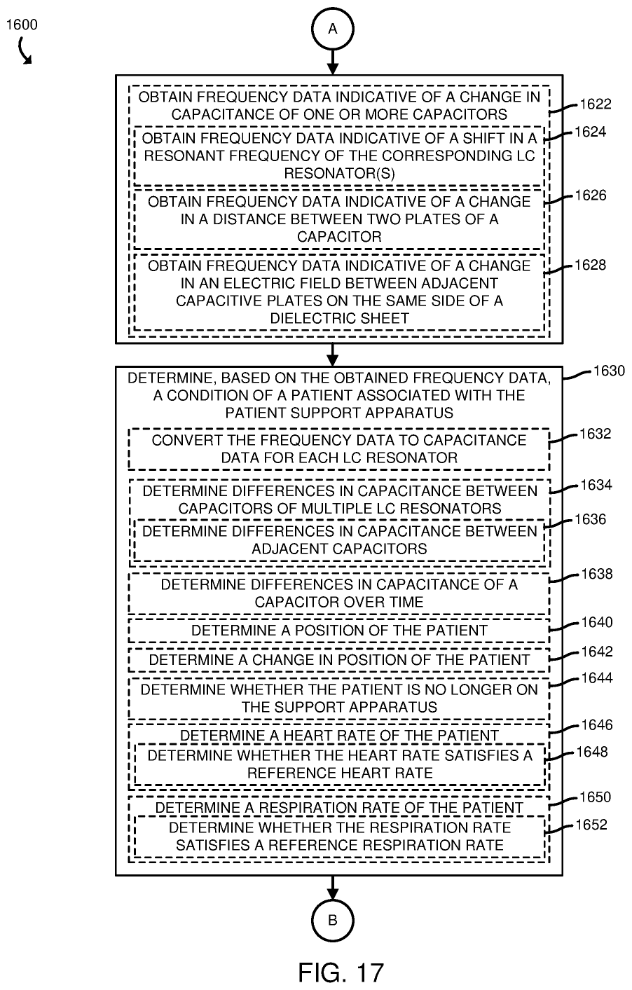

Referring now to FIG. 17, the mat 5 obtains frequency data indicative of a change in capacitance of one or more capacitors (e.g., each formed by a pair of plates 63, 64), as indicated in block 1622. In doing so, the mat 5 obtains frequency data indicative of a shift in a resonant frequency of the corresponding LC resonator(s), as indicated in block 1624. As indicated in block 1626, the mat 5 obtains frequency data indicative of a change in a distance between two plates of a capacitor. Additionally or alternatively, the mat 5 may obtain frequency data indicative of a change in an electric field (e.g., the fields 1400, 1500) between adjacent capacitive plates (e.g., plates 1402, 1404) on the same side (e.g., the upper side) of a dielectric sheet (e.g., the dielectric sheet 65), as indicated in block 1628.

Subsequently, and as indicated in block 1630, the mat 5 determines, based on the obtained frequency data, a condition of a patient associated with the patient support apparatus 1 (e.g., hospital bed). In doing so, the mat 5 converts the frequency data (e.g., from block 1604) to capacitance data (e.g., any data indicative of a capacitance) for each LC resonator of the mat 5, as indicated in block 1632. As described above, with reference to FIG. 7, the oscillation frequency (e.g., resonant frequency) of an LC resonator is a function of the capacitance of the capacitor in the LC resonator. The mat 5 may also determine differences in capacitance between capacitors of multiple LC resonators, as described above with reference to FIGS. 13-15 and as indicated in block 1634 of FIG. 17. In doing so, the mat 5 determines differences in capacitance between adjacent capacitors, as indicated in block 1636. As indicated in block 1638, the mat 5 may determine differences in capacitance of each individual capacitor over time as well (e.g., indicating that a patient has moved onto or off of the corresponding portion of the patient support apparatus 1).

In block 1640, the mat 5 determines a position of the patient (e.g., by comparing capacitance data of the multiple LC resonators and locations of their corresponding capacitors in the mat 5). Additionally, the mat 5 may determine a change in position of the patient (e.g., by comparing position data from block 1640 to earlier or subsequent position data), as indicated in block 1642. Relatedly, the mat 5 may determine whether the patient is no longer on the mat 5 (e.g., determine whether the LC resonators have an oscillation frequency consistent with the plates 63, 64 no longer being pressed closer together by the weight of a patient), as indicated in block 1644. As indicated in block 1646, the mat 5 may identify a time-varying capacitance associated with one or more of the LC resonators that is consistent with a heart rate (e.g., in terms of frequency and amplitude), and identify it as such. In doing so, and as indicated in block 1648, the mat 5 may determine whether the identified heart rate satisfies one or more reference heart rates (e.g., is within a reference range of heart rates that are defined as being normal for bed rest). Similarly, the mat 5 may identify a time-varying capacitance associated with one or more of the LC resonators that is consistent with a respiration rate (e.g., in terms of frequency and amplitude), and identify it as such, as indicated in block 1650. As indicated in block 1652, the mat 5 may determine whether the identified respiration rate satisfies one or more reference respiration rates (e.g., is within a range of respiration rates that are defined as normal for a patient on bed rest).

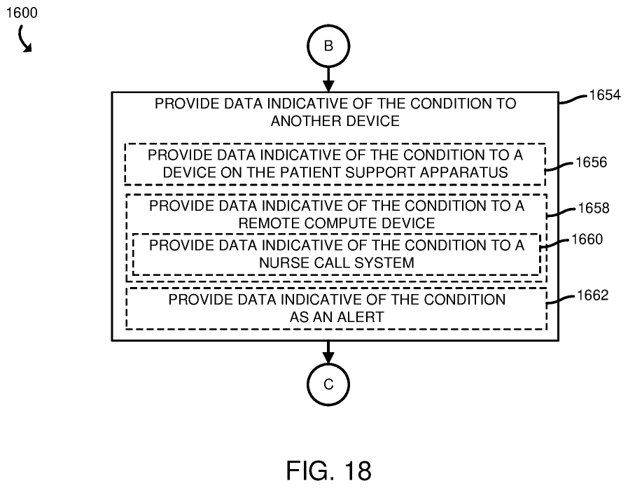

Subsequently, and referring now to FIG. 18, the mat 5 provides data indicative of the condition of the patient to another device, as indicated in block 1654. In doing so, the mat 5 may provide data indicative of the condition to a device on the patient support apparatus 1 (e.g., the control GUI device 22), as indicated in block 1656. Additionally or alternatively, the mat 5 may provide data indicative of the condition to a remote compute device, as indicated in block 1658. For example, the mat 5 may provide the data to one or more compute devices of a nurse call system (e.g., through a wired or wireless connection to the hospital data network), as indicated in block 1660. In providing the data, the mat 5 may provide the data as an alert (e.g., send an alert, such as a textual message or code indicative of the nature of the alert (e.g., patient has left the bed, low heart rate, high heart rate, low respiration rate, high respiration rate), a severity level of the alert, and/or other data indicating that a caregiver such as a nurse should attend to the patient), as indicated in block 1662. Afterwards, the method 1600, in the illustrative embodiment, loops back to block 1604 of FIG. 16 to continue to obtain frequency data from one or more of the LC resonator(s). Although the operations of the method 1600 are shown in FIGS. 16-18 in a particular order, it should be understood that the operations may be performed in a different order and/or concurrently (e.g., providing data indicative of the patient's condition to another device while concurrently obtaining additional frequency data from the LC resonator(s)).

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

1. A flexible mat configured to be placed between a patient and a patient support apparatus, for determining a condition of a patient based on LC resonator data, the flexible mat comprising:
a set of one or more LC resonators, wherein each LC resonator includes a capacitor and an inductor and is configured to oscillate at a frequency associated with a present capacitance of the capacitor; and
circuitry to:
measure an oscillation frequency of at least one of the LC resonators;
convert the measured oscillation frequency to digital frequency data that is indicative of the measured oscillation frequency; and
determine, based on the frequency data, at least one of a position, a heart rate, or a respiration rate of the patient.

2. The flexible mat of claim 1, wherein to obtain frequency data from at least one of the LC resonators comprises to obtain frequency data from a plurality of LC resonators and wherein to determine, based on the obtained frequency data, a position of the patient comprises to:
convert the frequency data to capacitance data for each LC resonator;
determine a difference in capacitance between capacitors of multiple LC resonators; and
determine the position of the patient as a function of the difference in the capacitance.

3. The flexible mat of claim 2, wherein to determine a difference in capacitance between capacitors of multiple LC resonators comprises to determine a difference in capacitance between adjacent capacitors.

4. The flexible mat of claim 1, wherein the circuitry is further to determine a difference in capacitance of one of the capacitors over time.

5. The flexible mat of claim 1, wherein the circuitry is further to determine whether the patient is located on the patient support apparatus.

6. The flexible mat of claim 1, wherein the circuitry is further to determine whether the heart rate satisfies a reference heart rate.

7. The flexible mat of claim 1, wherein the circuitry is further to determine whether the respiration rate satisfies a reference respiration rate.

8. The flexible mat of claim 1, wherein the circuitry is further to provide data indicative of the position, heart rate, or respiration rate of the patient to another device.

9. The flexible mat of claim 1, wherein to obtain frequency data comprises to obtain frequency data from each of multiple LC resonators having a set of capacitors formed by two arrays of conductive plates on opposite sides of a dielectric sheet.

10. The flexible mat of claim 1, wherein to obtain frequency data comprises to obtain frequency data from each of eight LC resonators.

11. The flexible mat of claim 1, wherein to obtain frequency data comprises to obtain frequency data from multiple LC resonators, each having a set of capacitive plates configured as a single ended grounded sensor.

12. The flexible mat of claim 1, wherein to obtain frequency data comprises to obtain frequency data from multiple LC resonators, each having a set of capacitive plates configured as a differential grounded sensor.

13. The flexible mat of claim 1, wherein to obtain frequency data comprises to obtain frequency data from multiple LC resonators, each having a set of capacitive plates configured as a single ended floating sensor.

14. The flexible mat of claim 1, wherein to obtain frequency data comprises to obtain frequency data from multiple LC resonators, each having a set of capacitive plates configured as a differential floating sensor.

15. The flexible mat of claim 1, wherein to obtain frequency data comprises to obtain frequency data indicative of a shift in a resonant frequency of one or more of the LC resonators.

16. The flexible mat of claim 1, wherein to obtain frequency data comprises to obtain frequency data indicative of a change in a distance between two plates of a capacitor of one of the LC resonators.

17. The flexible mat of claim 1, wherein to obtain frequency data comprises to obtain frequency data indicative of a change in an electric field between adjacent capacitive plates on the same side of a dielectric sheet.

18. A patient bed comprising:
a frame having a support deck;
a mattress; and
a flexible mat located between the support deck and the mattress, wherein the flexible mat comprises:
a set of one or more LC resonators, wherein each LC resonator includes a capacitor and an inductor and is configured to oscillate at a frequency associated with a present capacitance of the capacitor; and
circuitry to:
measure an oscillation frequency of at least one of the LC resonators;
convert the measured oscillation frequency to digital frequency data that is indicative of the measured oscillation frequency; and
determine, based on the frequency data, at least one of a position, a heart rate, or a respiration rate of the patient.

19. The patient bed of claim 18, wherein to obtain frequency data from at least one of the LC resonators comprises to obtain frequency data from a plurality of LC resonators and wherein to determine, based on the obtained frequency data, a position of the patient comprises to:
convert the frequency data to capacitance data for each LC resonator;
determine a difference in capacitance between capacitors of multiple LC resonators; and
determine the position of the patient as a function of the difference in the capacitance.

20. A method comprising:
measuring, by a flexible mat located between a patient and a patient support apparatus and having a set of one or more LC resonators, wherein each LC resonator includes a capacitor and an inductor and is configured to oscillate at a frequency associated with a present capacitance of the capacitor, an oscillation frequency of at least one of the LC resonators;
converting, by the flexible mat, the measured oscillation frequency to digital frequency data that is indicative of the measured oscillation frequency; and
determining, by the flexible mat and based on the frequency data, at least one of a position, a heart rate, or a respiration rate of the patient.
